(19) United States
(12) Patent Application Publication
(10) Pub. No.: US 2005/0152914 A1
(43) Pub. Date: Jul. 14, 2005

(54) Formulation of antigen

(75) Inventors: Robert Harris, Solna (SE); John Robertson, Stockholm (SE); Anders Essen-Moller, Stockholm (SE)

Correspondence Address: ROGER A. GILCREST, 250 WEST STREET, COLUMBUS, OH, 43216-7513, US

(73) Assignee: Diamyd, Inc.

(21) Appl. No.: 10/806,358

(22) Filed: Mar. 19, 2004

Related U.S. Application Data
(60) Provisional application No. 60/478,392, filed on Jun. 13, 2003.

(51) Int. Cl. A61K 39/00

(52) U.S. Cl. 424/184.1

(57) ABSTRACT

The present invention regards methods and formulations for diagnosis, prevention and treatment of disease. More particularly, the present invention teaches methods and formulations for diagnosis, prevention and treatment with antigen in autoimmune disease, allergy, rejection of transplants and cancer. Examples illustrate how the methods and formulations of the invention may be used for diagnosis and amelioration of autoimmune diabetes in which the 65 kd isotype of glutamic acid decarboxylase (GAD) is a major antigen.

### CROSS-REFERENCE TO RELATED APPLICATIONS

This application claims the benefit of U.S. Provisional Patent Application No. 60/415,494, filed on Oct. 2, 2002 and Ser. No. 60/478,392, filed Jun. 13, 2003, which are incorporated herein by reference.

The present invention regards methods and formulations for diagnosis, prevention and treatment of disease. More particularly, the present invention teaches methods and formulations for diagnosis, prevention and treatment with antigen in autoimmune disease, allergy, rejection of transplants and cancer. Examples illustrate how the methods and formulations of the invention may be used for diagnosis and amelioration of autoimmune diabetes in which the 65 kd isotype of glutamic acid decarboxylase (GAD) is a major antigen.

Lymphocytes play fundamental roles in disease defense and pathogenesis, and much effort is currently directed to defining both disease-promoting and disease-protective T cell responses in order to facilitate diagnosis and development of appropriate therapies. A variety of organ-specific inflammatory diseases have been dissected with respect to the relative roles of CD4+ and CD8+ cells. In the non-obese diabetic (NOD) mouse, a murine model for diabetes mellitus, transfer of both CD4+ and CD8+ T cell clones cause islet infiltration and destruction (Wong F. S., 1999). In mouse collagen-induced arthritis (CIA) reported data suggest that CD4+ and CD8+ T cells could independently promote the development of CIA (Tada, 1996). Although Myasthenia Gravis is thought to be an antibody-driven autoimmune disease affecting neuromuscular junctions, both CD4+ and CD8+ cells are reported to be important for development of experimental autoimmune myasthenia gravis (EAMG) (Zhang, 1996). When the autoimmune mechanisms in experimental autoimmune myocarditis (EAM) were dissected it was concluded that neither CD4+ nor CD8+ T cells were essential for disease (Penninger, 1993 #88). Therefore, in different experimental autoimmune models, CD4+ and CD8+ T cells may play different roles in pathogenesis.

It is known in the art that naive regulatory T cells develop into various subsets of T helper cells upon antigen stimulation depending not only on the antigen itself and/or the patient's genetic predisposition but also on the context in which the antigen is presented. For example, in the NOD mouse, stimulation with antigen in association with Complete Freunds Adjuvant induces a response, where proinflammatory Th1 cells dominate over those giving a humoral response to associated Th2 cells. In contrast, if the antigen is administered with Incomplete Freunds Adjuvant, the Th2 response will be enhanced. Some of the activated Th cells will in turn develop into longlived memory T cells committed to a specific immune response upon re-stimulation.

The prevailing cytokine profile and the activity of the immune system may vary from time to time within an individual; for example an ongoing virus infection may in many cases alert the cell-mediated arm of the immune defense system more than a bacterial infection. Therefore, encounter with an antigen administered as a part of antigen specific therapy may affect the immune system differently depending on its actual status at the time of administration.

It is an object of the present invention to present a method for selecting the route of administration and formulation for antigen-specific therapy—aiming at accomplishing a disorder appropriate immunomodulation.

It is a further object of the present invention to disclose non-toxic immunoneutral formulations capable of solubilizing hydrophilic as well, as hydrophobic protein antigens.

It is yet a further object of the present invention to disclose a formulation with the GAD antigen capable of ameliorating beta cell specific inflammation in the islets of Langerhans in man.

### SUMMARY OF THE INVENTION

In general terms, the present invention includes a method to stimulate the immune system in vitro with an antigen in a formulation without interfering with the current preprogrammed response of the immune system to that antigen by means of administering the antigen in an otherwise immunoneutral formulation.

The invention also includes a method to modulate an undesired current status of the immune system in an individual by: (a) evaluating the current immune response to an antigen in vitro according to the method referred to above; and (b) determining or deciding on a therapeutic formulation suitable for specific immunomodulation away from of the individual's current unwanted immune response.

The invention also includes a formulation that is immunoneutral to the immune system of an individual and capable of solubilizing hydrophobic antigens, including one wherein the immunoneutral hydrophobic solvent is species specific Serum Albumin.

The formulation may optionally include an antigen for stimulation of the immune system.

The formulation for antigen specific downregulation of inflammatory responses of the present invention may also comprise at least two of the ingredients antigen and alum. Preferably, the antigen used in the present inventions includes at least one from the group of ICA512 (IA2), ICA512B (IA2B), insulin, insulin B-chain, proinsulin, Hsp60, Hsp65, P277, ICA69, Glima38, GAD 65, GAD67, SOX13, Imogen 38, Sulfatide, MBP, MOG, Collagen II, 21-Ohase, TPO, allergens, transplant antigens, cancer antigens, or parts, peptides or altered peptide ligands thereof.

The invention also includes a kit for evaluation of lymphocyte reaction to antigen comprising a formulation according to the present invention, as well as a therapeutic composition comprising a formulation according to the present invention.

It is preferred that the therapeutic composition according to the present invention provide concentrations of antigen for in vitro stimulation typically is between 1-100 micrograms per ml, preferably between 5-40 micrograms per ml.

The invention also includes a therapeutic composition as described above where the subcutaneous administration of antigen for in vivo stimulation typically is between 0.1 micrograms per ml and 100 micrograms per ml, or where intravenous administration of antigen for in vivo stimulation typically is between 0.01-5 mgs/kg.

a. The invention also includes a method to stimulate the immune system in vitro with an antigen in a formulation without interfering with the current preprogrammed response of the immune system to that antigen by means of administering the antigen in an otherwise immunoneutral formulation.

Also forming part of the invention is a method to modulate an undesired current status of the immune system in an individual by: (a) evaluating the current immune response to an antigen in vitro according to the method of the present invention as described herein; and (b) determining or deciding on a therapeutic formulation suitable for specific immunomodulation away from of the individual's current unwanted immune response.

The formulation used is one that is immunoneutral to the immune system of an individual and capable of solubilizing hydrophobic antigens, such as where an immunoneutral hydrophobic solvent is used, e.g., a species specific Serum Albumin.

This formulation preferably includes an antigen for stimulation of the immune system, such as a formulation for antigen specific downregulation of inflammatory responses comprising at least two of the ingredients antigen and alum. The antigen preferably includes at least one from the group of ICA512 (IA2), ICA512B (IA2B), insulin, insulin B-chain, proinsulin, Hsp60, Hsp65, P277, ICA69, Glima38, GAD 65, GAD67, SOX13, Imogen 38, Sulfatide, MBP, MOG, Collagen II, 21-Ohase, TPO, allergens, transplant antigens, cancer antigens, or parts, peptides or altered peptide ligands thereof.

The concentrations of antigen and the amounts used for subcutaneous administration may be as described above.

### DETAILED DESCRIPTION OF THE INVENTION

In order to diagnose, prevent or treat autoimmune disease, transplant rejection, or cancer it is of increasing interest to expose various antigens to naive and activated lymphocytes in a case appropriate context, such that the lymphocyte response results in production of cytokines that promote the overall immune response to the particular antigen in a desired way. In transplant rejection and organ specific autoimmune disease, such as for example type I diabetes, downregulation of inflammation in or around the specific organ is desired whereas upregulation of specific cytotoxic lymphocytes is desired in treatment of cancer.

When stimulating lymphocytes with antigen, it is of particular importance that, apart from endotoxin levels being low, the formulation itself is immunoneutral while being able to keep an antigen in solution. This is so that the formulation itself does not stimulate the immune system in a particular direction.

As the status of an individual's immune system may vary from time to time, exposure to an antigen may give a different response from the immune system on different occasions. For example, it can be speculated that as viral disease activates the cell-mediated arm of the immune system with a characteristic Th1 cytokine profile, administration of unadjuvanted endogenous antigen may give rise to autoimmune disease in a susceptible individual. Once a lymphocyte response to an antigen solubilized in an immunoneutral formulation is defined at a particular time, the route of administration and formulation for an antigen specific therapeutic can be decided upon according to the method of the present invention—aiming at accomplishing appropriate immunomodulation for a given disorder.

The formulations of the present invention disclose non-toxic immunoneutral formulations capable of solubilizing hydrophilic as well as hydrophobic protein antigens. In addition the present innovation presents a formulation of GAD capable of downregulating IFN-gamma production in individuals with GAD reactive T cells—indicating a downregulation of beta cell destruction in the islets of Langerhans, and of inducing a non-inflammatory response to the GAD antigen for prevention and therapy of diabetes.

### EXPERIMENT I

In human diabetes, a number of GAD specific T cell clones have been identified in the peripheral blood of type 1 diabetes patients (Roep ref). As a starting point for such cloning, and as an integral part of clinical immunological testing, it is critical to be able to re-stimuiate GAD-specific T cells derived from peripheral blood in vitro. The use of GAD in assays for in vitro re-stimulatation of T cells to elicit cytokine production and cellular proliferation, is an important and problematic issue that has led to the establishment of an International T Cell Workshop whose primary aim is to establish sources of diabetes-relevant antigens and appropriate protocols for their use. GAD has been expressed as a recombinant protein in bacteria, insect cells and in yeast. The consensus from this Workshop is that recombinant Diamyd™ GAD (commercially available from Diamyd, Inc. of Raleigh, N.C.), produced by baculovirus expression in insect cells, is appropriate and useful for in vitro T cell assays (Roep, 1999 #262; Peakman, March 2002).

However, the GAD preparations are still not ideal, and the experience of the T Cell Workshop can be summarized as: i) GAD in buffer comprising reducing agents and detergents is toxic to T cells; and ii) dialysis of GAD reduces toxicity but increases risk of precipitation.

In order to improve the suitability of proteins such as GAD or MOG (an antigen associated with multiple sclerosis) for human T cell work, new formulations were devised and tested for efficacy in different T cell assays. The premise for this formulation was to 1) reduce the toxicity inherent in the buffer solutions; 2) without by itself stimulating or directing a response from the immune system; and 3) while allowing full solubility of the protein, which is usually lost on direct buffer exchange to PBS. The formulation was unexpectedly achieved through addition of Human Serum Albumin (HSA) during the process of buffer exchange to cell culture medium (RPMI).

Although not limited to the theory of operation of the invention, it is believed that the addition of an immunogenetically ‘neutral’ protein would allow preferential interaction of the protein as its buffer is replaced with cell culture medium, preventing the protein molecules interacting with each other and thus preventing precipitation.

a. Cell culture medium was selected for buffer exchange, because this is the assay medium used for in vitro immunological assays, Human serum albumin (HSA) was selected as an example of an immunologically neutral protein, as this is a major component of human blood proteins and also of complete cell culture used in vitro assays. It would thus not cause induction of any type of immune response itself per se, and would therefore not itself impact on the reactions or functions of the T cells in the assay.

The process of protein precipitation occurs due to protein molecules developing a higher charge affinity to each other than to the surrounding medium, leading to protein-protein aggregation that becomes apparent as an insoluble precipitate. This occurs for example during buffer exchange of GAD in Diamyd™ buffer (commercially available from Diamyd, Inc.) to PBS. The electrostatic charge of GAD changes as the detergent and reducing agent in the Diamyd buffer is replaced by PBS.

Whereas the approach did not work for the MOG antigen which precipitated when it in a pH3 buffer was dialysed against HSA, the GAD antigen stayed perfectly in solution during similar treatment.

Optimisation of the formulation of T cell GAD was based on the solubility and T cell stimulatory capacities of different formulations studied in vitro. The concentrations of GAD and of HSA were varied in two different experimental series:

i. SERIES 1. SOLUBILITY TESTING

| SAMPLE | GAD conc | HSA conc | Ratio | |
|---|---|---|---|---|
| I.D. | mg/ml | mg/ml | GAD:HSA | Dialysis buffer |
| A | 6.5 | 3.25 | 2:1 | RPMI |
| B | 6.5 | 6.5 | 1:1 | RPMI |
| C | 2 | 1 | 2:1 | RPMI |
| D | 1 | 1 | 1:1 | RPMI |
| E | 1 | 0.5 | 2:1 | RPMI |
| F | 1 | 0 | 1:0 | RPMI |
| Control | 2.5 | 1 | 2.5:1 | RPMI |

a. SERIES 2. T CELL STIMULATION

| b. | SAMPLE | GAD conc | Albumin conc | | Ratio | Dialysis |
|---|---|---|---|---|---|---|
| i. | I.D. | mg/ml | mg/ml | | GAD:HSA | Buffer |
| ii. | 1 | 1 | I | (HSA) | 1:1 | RPMI |
| iii. | 2 | 1 | 0 | | 1:0 | RPMI |
| iv. | 3 | 0 | 1 | (HSA) | 0:1 | RPMI |
| v. | 4 | 1 | 0 | | 1:0 | Diamyd |
| | | | | | | Buffer |
| vi. | 5 | 1 | 20 | (HSA) | 1:20 | RPMI |
| vii. | 6 | 1 | 0 | | 1:0 | ddH2O |
| viii. | 7 | 0 | 20 | (HSA) | 0:20 | RPMI |
| ix. | 8 | 1 | 1 | (MSA) | 1:1 | RPMI |
| x. | 9 | 0 | 1 | (MSA) | 0:1 | RPMI |

Formulations were assessed for precipitation by visual inspection and were additionally assessed for structural integrity and immunogenicity by SDS PAGE and western blotting with both N- and C-terminal specific monoclonal antibodies. Different T cell assays endpoints were measured as follows:

| CELLULAR SOURCE | ASSAY |
|---|---|
| human PBMC | proliferation |
| human PBMC | IFN-γ cell awface FACS |
| humn T cell lines | proliferation |
| human T cell lines | proliferation |
| NOD mouse PBMC | IFN-γ ELISPOT |
| NOD mouse T cell lines | proliferation |
| human serum | antibody radio immunoassay |

The concentration of the GAD dialysed is important. The most efficient GAD concentration was 1 mg/ml, with no detectable precipitate. At high concentrations (i.e. 6.54 mg/ml) there is still appreciable precipitation after dialysis.

The ratio of HSA:GAD is important. The most efficient HSA:GAD ratio was 1:1, with which there was no detectable precipitate. At more than 1:1 (HSA:GAD) there is increased precipitation, irrespective of the concentration of GAD in the sample.

Dialysis of GAD with RPPM, with or without the inclusion of HSA, gave stimulation of T cells in proliferation, cytokine capture and ELISPOT analyses. Irrespective of whether human PMBC or defined T cell lines, or mouse PBMC or defined mouse T cell lines were used in these assays, the dialysed GAD preparations performed better than the original non-dialysed counterpart.

The solubility of GAD greatly affected its T cell stimulatory ability, the least soluble preparations stimulating less efficiently than their soluble counterparts.

Based on the superior stimulation of T cells, the final formulation of tcGAD was that GAD65 should be dialysed at a concentration of 1 mg/ml with 1 mg/ml HSA against RPMI for use in in vitro T cell assays.

### EXPERIMENT 2

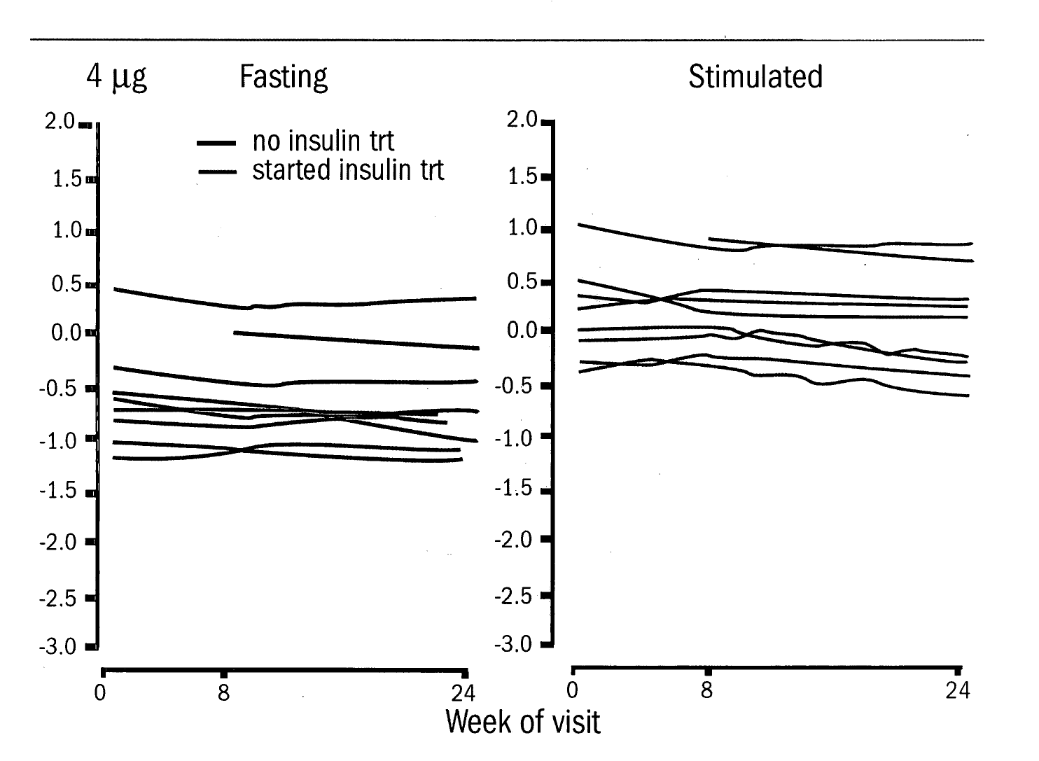

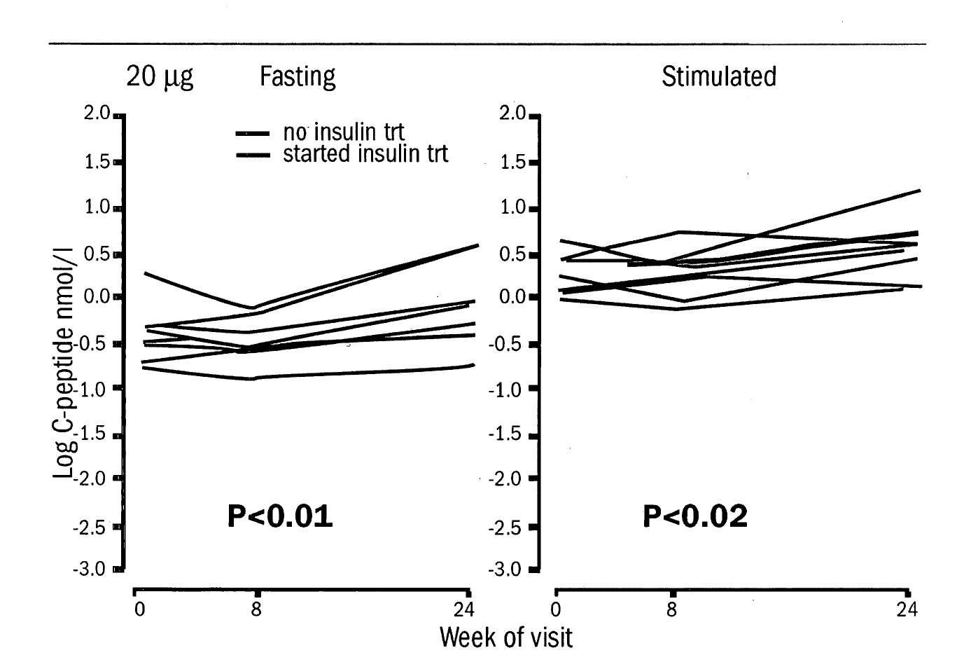

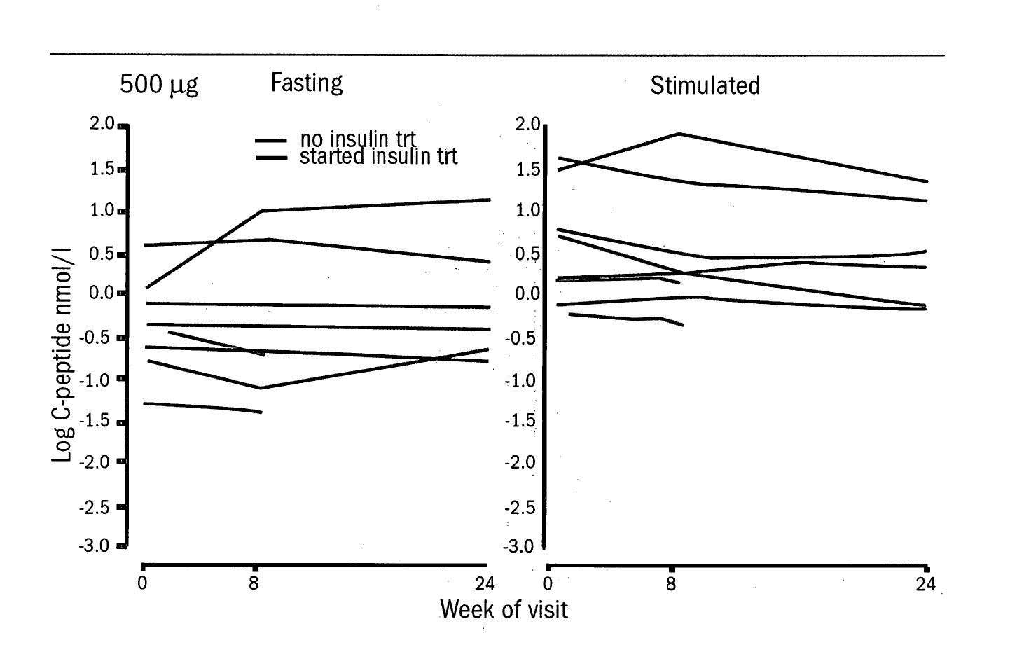

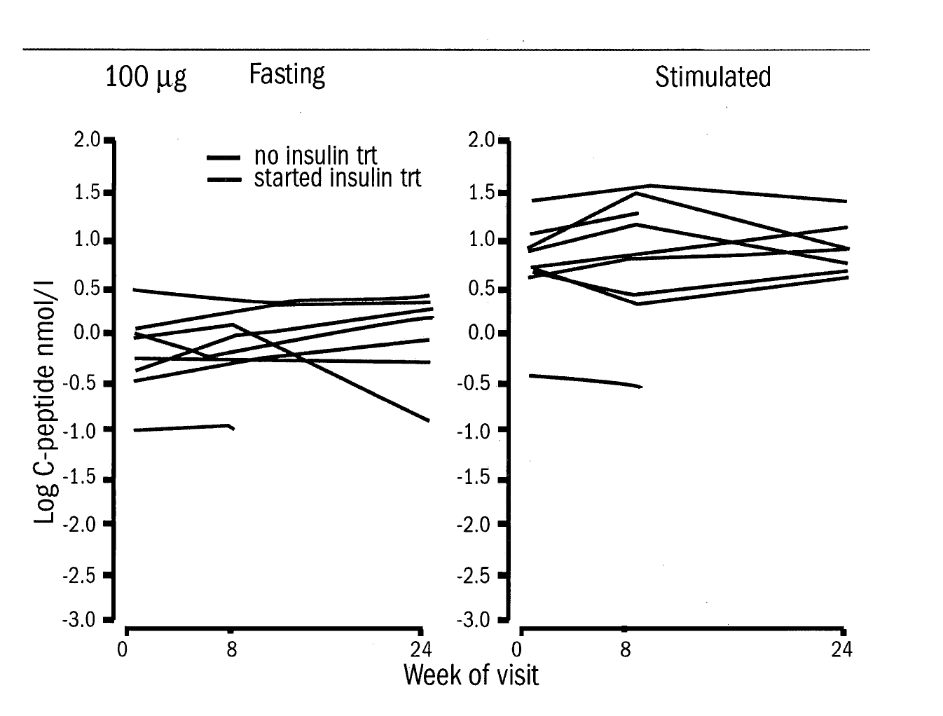

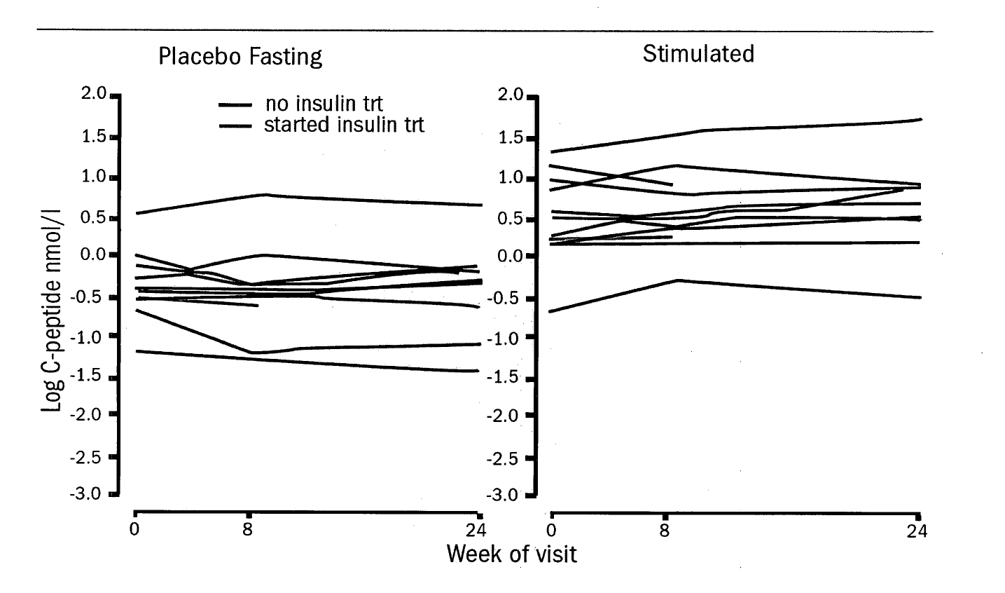

Glutarmic acid decarboxlase (GAD65) is an autoantigen proposed to be a major target of autoimmunity during initiation and maintenance of the inflammatory process leading to beta cell destruction, mid insulin dependency in man. T cells from type-2 diabetes patients with GAD antibodies were stimulated in vitro with GAD formulated in HSA before and after in vitro subcutaneous administration of GAD formulated in alum. As HSA is a common soluble protein in man, the GAD-HSA formulation was intended not to modify the status of the immune system. Stimulation with tetanus toxoid was used as control. IFN-gamma was measured with cytokine secretion assays. As is shown in FIG. 1 IFN-gamma secretion was dramatically reduced upon GAD-HSA in vivo stimulation after subcutaneous in vivo administration of GAD-alum. The effect was persistent over at least four weeks and after a subsequent subcutaneous boost.

Analysis of Phase II Diamyd Study After Unblinding Kristen Lynch Biostatistican

This analysis extents on the summary measure statistics performed by Chiltern. They summarized each metabolic measurement GAD Ab level by group and time. The aim of this analysis is to calculate the change in metabolic or GAD level for each person within each of the 4 dose groups and 1 placebo group, and analyze these changes in measurement over time. Change in measurement is the week 24 level of the relevant variable minus the baseline or day 1 level. The first part of the group differed from zero. This was done by calculating difference in levels for every subject and examining whether mean and median change in levels differed from zero. Next, the mean changes in levels for group 1 to 4 were individually compared to the mean change in levels for the placebo group. Both mean and median levels are presented in tables except for GAD levels which is a highly skewed distribution. Only median is present when describing change in GAD levels. Paired t-test examined for a change in levels differed between groups. When mean and median differed the appropriate non-parametric test was used to describe difference in medians. P-value is the probability of observing a result shown if there really is no difference. A p-value less than 0.05 was considered significant.

Attached are graphs of log C-peptide over time for each individual. (log C-peptide was used instead of C-peptide because log is normally distributed in the normal population, there is also a few outliers which make it difficult to put all the individuals on the same scale and C-peptide is less accurate at high values. In particular the conversion from C-peptide to Plasma C-peptide was less accurate for C-peptide values above 2.1 n/mol. Using the log transformation limits the influence of measurement error).

The ID numbers in the dose groups are the new numbers supplied by Chiltern. The ID numbers in the placebo group are the numbers given but with 40 added on, i.e., P-1 is P-41 (it made it easier for me to draw graphs). The dotted line indicates that patients that started on insulin after 24 weeks. There were four patients who started insulin in group 1, one in group 2, one in group 3, and two in group 4. One patient started insulin in the placebo group. Looking at the graphs it was evident patients who were on insulin were the same patients with the lowest C-peptide at baseline (day 1). Starting insulin is therefore not a good indication of how effective the dose was and was not examined further. However it should be noted that in does groups 100 and 500 ug and in the placebo group, there was no week 24 C-peptide measurement for patients who started insulin treatment. Yet in the 4 ug dose all 4 insulin patients were observed at week 24. This in itself can great a difference in change between the groups. Note these 4 patients were part of the handful of patients with a stimulated log C-peptide below 0.0 and who were followed for the entire 24 weeks. These patients showed the steepest decline in stimulated C-peptide after 8 weeks within the group of patients receiving 4 ug of the drug.

These plots show first of all no sudden decline in C-peptide. From a safety perspective this indicates the drug does not interfere negatively with the production of endogenous insulin. There was however one outlier in change of C-peptide. Patient 25 (new ID) 42 (old ID) showed a sudden drop in pre-sustacal (fasting) C-peptide after 8 weeks. Interestingly, this patient had the highest BMI at the start of the study 39 Kg/m2 and remained at 39 Kg/m2 till week 24. His GAD level remained only borderline positive (32-48 U/ml) till week 24 after which it became negative.

The tables below examine the change in log fasting C-peptide, log post-sustacal (stimulated) C-peptide, glucose and HbA1c. These measurements were used to measure the end result of administering the GAD65 vaccine.

TABLE 1
Mean (median) change in pre(fasting) and post(stimulated) sustacal C-peptide

| | | Fasting | | | Stimulated | | |
|---|---|---|---|---|---|---|---|
| | | Log C-peptide (n/mol.) | | | Log C-peptide (n/mol) | | |
| | | Change from | 1 | 2 | Change from | 1 | 2 |
| Group | n | Day 1-week 24 | p-value | p-value | Day 1-weweek 24 | p-value | p-value |
| Placebo | 13 | −0.02 | 0.75 | N/A | +0.14 | 0.11 | N/A |
| | | (+0.08) | | | | | |
| 4 ug | 8 | −0.11 | 0.09 | 0.57 | −0.17 | 0.007 | 0.002 |
| | | (+0.10) | | | (−0.19) | | |
| 20 ug | 8 | +0.37 | 0.01 | 0.01 | 0.25 | 0.02 | 0.27 |
| | | (+0.30) | | | (+0.17) | | |
| 100 ug | 8 | +0.07 | 0.71 | 0.56 | +0.06 | 0.50 | 0.44 |
| | | (+0.12) | | | (0.00) | | |
| 500 ug A | 8 | +0.01 | 0.90 | 0.87 | −0.24 | 0.13 | 0.008 |
| | | (+0.06) | | | (−0.27) | | |
| 500 ug B | 8 | +0.28 | 0.53 | 0.17 | −0.66 | 0.08 | <0.001 |
| | | (−0.01) | (0.99) | | (−0.45) | | |

Test whether change is different from zero
Test whether change is different from change in Placebo group when mean and median differ (>.1), p-values are give for both.
A = 2 Doses,
B - more than 2 Doses

TABLE 2
Mean (median) change in fasting glucose and HbA1c

| | Fasting Glucose | | | HbAlc % | | |
|---|---|---|---|---|---|---|
| | Change from | 1 | 2 | Change from | 1 | 2 |
| Group | Day 1-week 24 | p-value | p-value | Day 1-week 24 | p-value | p-value |
| Placebo | +0.85 | 0.28 | N/A | +0.30 | 0.01 | N/A |
| | (+0.90) | | | (+0.25) | | |
| 4 ug | +1.34 | 0.35 | 0.72 | +0.31 | 0.22 | 0.97 |
| | (+0.20) | | | (+0.20) | | |
| 20 ug | −0.74 | 0.52 | 0.25 | −0.31 | 0.40 | 0.059 |
| | (−0.05) | (0.74) | | (−0.05) | | |
| 100 ug | −0.25 | 0.59 | 0.41 | −0.08 | 0.61 | 0.21 |
| | (0.00) | | | | | |
| 500 ug A | −2.0 | 0.30 | 0.08 | −0.16 | 0.75 | 0.22 |
| | (−1.7) | | | (+0.20) | | |
| 500 ug B | 0.85 | 0.11 | 0.99 | +0.45 | 0.56 | 0.78 |
| | (+0.45) | | | | | |

p-value test whether mean is different from zero
p-value test whether change is a different from change in Placebo group
When mean and median differ (>.5), p-value are given for both.
A = 2 Doses,
B = more than 2 Doses

Both the means are medians are useful summary measures of centrality for small sample groups here. The median indicates the middle value and a comparison with the mean shows if there are any patients who have a change in level more extreme than other patients in the group.

A comparison of means and median of C-peptide within each group show both to be similar except perhaps in the smaller 500 ug subgroups. Glucose and HbA1c show mean and median to differ in the 20 ug group. One or two patients show a faster decline in glucose and HbA1c and thus much improved metabolic control compared to the change in other patients within the group.

Stimulated C-peptide appears to decline in the 4 ug and 500 ug groups and increase in the 20 ug group. Fasting C-peptide increases only in the 20 ug groups. A comparison of groups with placebos show the increase in fasting C-peptide in 20 ug group to be significantly different from the placebo group (p-value=0.01). In contrast Stimulated C-peptide declines faster in the 4 ug and 500 ug groups compared to the placebo group. However, it should be noted that the stimulated C-peptide appears to increase in the placebo group although not significantly. This is somewhat surprising as the HbA1c goes higher and is the only group to show an increase in HbA1c. With worsening metabolic control you might expect endogenous insulin to have decreased. Figure E shows one placebo showing a steady increase in stimulated C-peptide over time (and appears to be an outlier). Despite this increase in their endogenous insulin production, glucose increases from 7.3 to 8.0 and their HbA1c from 5.2 to 5.3 contrary to what you would expect from an improving patient. Even though there is no significant increase in stimulated C-peptide in the 20 ug group compared to the placebos, HbA1c shows signs of improved metabolic control compared with the same group (p-value=0.059).

Although these results have observed some improvement of metabolic parameters within the 20 ug group, the result is only an association. This is a study which is primarily used to show safety of the drug and is not designed to show the effect of the drug. However we can explore for further evidence of an effect by examining if there is an association between the drug and any intermediate changes and whether any intermediate changes is associated with improvement of metabolic control. With this in mind I decided to examine if the groups are associated with an increase in GAD levels over time and whether the same patients who show an increase in GAD levels show an increase in endogenous insulin production or a decrease in HbA1c and fasting glucose. An increase in GAD levels over an increase in GAD levels in the placebo group might indicate the drug is having some effect. Table 3 looks at change in GAD levels over time. Values are expressed as GAD level relative to GAD level when they entered the study (day zero). With the GAD distribution being highly skewed, this relative proportion made the comparison of change in GAD levels between patients or groups more meaningful.

A first glance at the graphs showed only an increase in GAD levels in patients who are considered to have a GAD level between 32 and 48 are considered to be borderline positive and did not show an increase in GAD over the 12-24 week period. This is important to consider as borderline positive may indicate another parameter involved but not measured, that may be involved with the development of type 1 diabetes. Thus the table below looks at borderline positive and positive-positive patients.

TABLE 3
Mean (GAD level/GAD level) at day 0

| | | | Day | Week | Week | Week | Week |
|---|---|---|---|---|---|---|---|
| Group | n | | 1 | 4 | 5 | 8 | 24 |
| Placebo | 5 | Borderline + | 0.65 | 0.64 | 0.64 | 0.62 | 0.65 |
| | 8 | +/− | 1.08 | 1.03 | 1.02 | 1.31 | 1.70 |
| 4 ug | 0 | Borderline + | — | — | — | — | — |
| | 9 | +/− | 1.04 | 0.98 | 1.11 | 1.28 | 1.37 |
| 20 ug | 3 | Borderline + | 1.02 | 1.01 | 1.07 | 1.03 | 0.77 |
| | 5 | +/− | 0.94 | 1.09 | 1.17 | 1.30 | 1.18 |
| 100 ug | 5 | Borderline + | 0.83 | 0.83 | 0.81 | 0.83 | 0.54 |
| | 4 | +/− | 0.80 | 1.68 | 1.88 | 3.05 | 3.06 |
| 500 ug A | 1 | Borderline + | 0.95 | 2.58 | 41.5 | 20.95 | — |
| | 4 | +/− | 1.18 | 3.73 | 4.59 | 7.02 | — |
| 500 ug B | 1 | Borderline + | 0.65 | 0.61 | 0.81 | 1.00 | — |
| | 2 | +/− | 1.19 | 1.08 | 0.96 | 1.06 | — |

Borderline = GADAb U/ml < = 0.48
+/+ GADAb U/ml > 0.48

If values are multiplied by 100 that can be interpreted as the percent of GAD value at baseline. Eg 1.31 at week 8 is 131% of the baseline GAD level or there is a 31% increase in GAD kevek by week 8.

Table 3 shows the distribution borderline positive within each group. None were borderline positive in group 1, but 3 were in group 2, 5 in group 3, and 2 in group 4. The high number of borderline positive in group 3 might indicate a less aggressive GAD auto-immune group.

Except for group 4 were only two patients were borderline positive, none of the other borderline positive patients showed an increase in GAD level. However in the above 48 groups a slight steady increase was seen in groups 1 and 2 and in placebos and more aggressive increase in group 3 and 4. Although these are mean values each group was homogenous or showed little variation.

Thus there was an association between increase in GAD and dose group and the association was dependent on the level of GAD on entry into the study.

Metabolic parameters are examined again but divided by whether or not the patient had borderline GAD 65 levels.

To prevent listing out another range of tables, the below only gives the p-value for change in measurement relative to change in Placebo group.

TABLE 4
Change in fasting and stimulated C-peptide in dose
groups relative to change in placebo group for borderline
and non borderline positive patients.

| | Fasting | | | | Stimulated | | | |
|---|---|---|---|---|---|---|---|---|
| | Log C-peptide (n/mol.) | | | | Log C-peptide (n/mol) | | | |
| | GAD < 48 | | GAD > 48 | | GAD < 48 | | GAD > 48 | |
| Group | p-value | | p-value | | p-value | | p-value | |
| Placebo | 0.08 | Ref. | −0.08 | ref. | 0.24 | ref. | 0.05 | ref. |
| 4 ug | — | — | 0.04 | 0.78 | — | — | −0.22 | 0.02 |
| 20 ug | 0.17 | 0.63 | 0.53 | <0.001 | 0.05 | 0.84 | 0.18 | 0.09 |
| 100 ug | −0.17 | 0.64 | 0.38 | 0.02 | −0.22 | 0.26 | 0.08 | 0.57 |
| 500 ug A | −0.26 | 0.62 | 0.19 | 0.327 | −0.52 | 0.11 | −0.27 | 0.05 |
| 500 ug B | 0.35 | 0.40 | 0.06 | 0.801 | −0.56 | 0.02 | −0.50 | 0.006 |

The values in the placebo row are the actual change. All other values in the column are compared to this reference change.

Table 4 shows how the level of GAD on day 1 may be indicator of whether the treatment will work. Within the GAD>48 group, where a majority show an increase in GAD levels over time, a significant increase in C-peptide is seen in the 20 ug and the 100 ug groups.

The HbA1c results confirm that the GAD>48 are not. All four of these patients showed a significantly increase in GAD levels compared to other patients in the placebo group who show only a steady small increase in GAD levels.

It is interesting to see how the higher dose groups in the GAD>48 patients have a greater decrease in fasting glucose compared to placebos GAD>48 patients. The higher GAD placebos showing an increase of 1.92 in fasting glucose while the 4 ug and 20 ug groups show no change (but in comparison a decrease) and 500 ug group who have a GAD>48 on day 1 and show a doubling of GAD over time show a significant decrease (p-value=0.05) compared to the placebo GAD>48 patients.

It can of course be argued that all significant values are due to change in the placebo group. The change in glucose HbA1c and C-peptide within the GAD>48 placebos is what one might expect from a group of high GAD positive patients left untreated. A decline of glucose (not significant) and a increase in C-peptide is not what you would expect (as seen in the borderline positive) and is more consistent with a non auto-immune disease.

TABLE 5
Change in fasting glucose and HbA1c in dose groups
relative to change in placebo group for borderline
and non-borderline positive patients.

| | Fasting Glucose | | | | HbAlc % | | | |
|---|---|---|---|---|---|---|---|---|
| | GAD < 48 | | GAD > 48 | | GAD < 48 | | GAD > 48 | |
| Group | p-value | | p-value | | p-value | | p-value | |
| Placebo | −1.02 | Ref. | 1.92 | ref. | 0.08 | ref. | 0.46 | ref. |
| 4 ug | — | — | −0.58 | 0.71 | — | — | −0.14 | 0.60 |
| 20 ug | 2.24 | 0.17 | −1.15 | 0.53 | −0.81 | 0.17 | −0.51 | 0.11 |
| 100 ug | 1.18 | 0.41 | −2.70 | 0.18 | −0.02 | 0.96 | −0.73 | 0.04 |
| 500 ug A | −1.38 | 0.57 | −3.83 | 0.05 | −1.89 | 0.04 | −0.21 | 0.55 |
| 500 ug B | 1.72 | 0.47 | −0.92 | 0.78 | −0.18 | 0.84 | 0.54 | 0.37 |

The values in the placebo row are the actual change. All other values in the column are compared to this reference change.

### CONCLUSION

The 20 ug group show an improvement in metabolic parameters but the dug may be most effective in patients who have more than a borderline GAD level at the start of the study. Five of the nine patients had only borderline values of GAD in group 3 and this might explain the little change in metabolic parameters overall within this group. Restricting to the remaining GAD positive patients did show an association between the dose level and an improvement in metabolic control.

Clinical trials resulted in a positive outcome with the GAD-based diabetes vaccine. The results may lead to a new treatment to prevent type 1 diabetes for which currently there is no cure.

Results of the phase II study has been tremendously successful. Not only is it now shown that the instant vaccine can be safely administrated in a wide range of doses, but a clear and significant positive effect (P=0.01) of the vaccine was found at one of the dose levels six months from first vaccination. It is also important to note that the trial was conducted to the highest standards which adds further weight to its results.

In type 1 diabetes, the immune system mistakenly destroys the insulin-producing cells in the pancreas in an autoimmune attack. Over time, this attack leads to a lack of insulin, the hormone that controls blood sugar levels. People with type 1 diabetes must inject insulin daily.

In type 2 diabetes, patients normally continue to produce their own insulin but are less sensitive to it. Therefore these patients may be treated with tablets to increase their sensitivity to insulin. A large group (10%) of the type 2 diabetes patients have antibodies to GAD. These patients are called LADA and suffer from a similar autoimmune attack as the type 1 diabetes patients, which leads to the need for insulin injections.

The phase II clinical trial was conducted by vaccinating patients with recently diagnosed LADA. The GAD-vaccine successfully improved these patients' ability to make insulin over a six-month period, compared with patients who received a placebo.

The trial shows that the vaccine is safe and that it is possible to inhibit the autoimmune attack on the cells that make insulin, thereby slowing the progression of the disease.

The vaccine to prevent type 1 diabetes arose from experiments with diabetes prone-mice that were protected from developing the disease by tolerizing them to GAD.

The phase II trial was conducted on 47 diabetes patients with the GAD-based vaccine at the UMAS hospital in Malmoe and St. Gorans Hospital in Stockholm, Sweden. The patients were randomly divided into four groups with 12 patients in each group. Each patient received on first injection of the instant vaccine followed by at least one boost injection four weeks after. Nine patients in every group received active drug whereas three received placebo. The groups received different doses of the vaccine ranging from 4 to 500 micrograms per dose. All patients visited the hospitals 10 times during this six-month study, and detailed clinical, immunological as well as neurological investigations showed no safety concerns at the administered dose levels.

The study results show that the diabetes vaccine significantly improves the serum C-peptide levels both at fasting (P=0.01) and after meals (P=0.02) at one of the doses.

The instant vaccine appears to be effective when given to people with an advanced disease and it appears that it will be highly effective when given at earlier stages of the disease process. We now know that type 1 diabetes takes years to develop and that we can detect people who are at early stages of the disease process by testing for GAD autoantibodies in their blood.

The first application for the instant vaccine is in older patients with adult onset diabetes with GAD antibodies since this patient group progress to full insulin dependence within a few years.

1. A method to stimulate the immune system in vitro with an antigen in a formulation without interfering with the current preprogrammed response of the immune system to that antigen by means of administering the antigen in an otherwise immunoneutral formulation.

2. A method to modulate an undesired current status of the immune system in an individual by:
a) evaluating the current immune response to an antigen in vitro according to the method of claim 1.
b) deciding on a therapeutic formulation suitable for specific immunomodulation away from of the individual's current unwanted immune response.

3. A formulation that is immunoneutral to the immune system of an individual and capable of solubilizing hydrophobic antigens.

4. A formulation according to claim 3 where immunoneutral hydrophobic solvent is species specific Serum Albumin.

5. A formulation according to claim 4 including an antigen for stimulation of the immune system.

6. A formulation for antigen specific downregulation of inflammatory responses comprising at least two of the ingredients antigen and alum.

7. A formulation according to claim 5 or 6, where the antigen is of at least one from the group of ICA512 (IA2), ICA512B (IA2B), insulin, insulin B-chain, proinsulin, Hsp60, Hsp65, P277, ICA69, Glima38, GAD 65, GAD67, SOX13, Imogen 38, Sulfatide, MBP, MOG, Collagen II, 21-Ohase, TPO, allergens, transplant antigens, cancer antigens, or parts, peptides or altered peptide ligands thereof.

8. A kit for evaluation of lymphocyte reaction to antigen comprising a formulation according to claim 7.

9. A therapeutic comprising a formulation according to claim 7.

10. A therapeutic according to claim 9 where concentrations of antigen for in vitro stimulation typically is between 1-100 micrograms per ml.

11. A therapeutic according to claim 9 where concentrations of antigen for in vitro stimulation typically is between 5-40 micrograms per ml.

12. A therapeutic according to claim 9 where the subcutaneous administration of antigen for in vivo stimulation typically is between 0.1 micrograms per ml.

13. A therapeutic according to claim 9 where intravenous administration of antigen for in vivo stimulation typically is between 0.1-5 mgs/kg.

14. A method to stimulate the immune system in vitro with an antigen in a formulation without interfering with the current preprogrammed response of the immune system to that antigen by means of administering the antigen in an otherwise immunoneutral formulation.

15. A method to modulate an undesired current status of the immune system in an individual by:
a) evaluating the current immune response to an antigen in vitro according to the method of claim 1.
b) deciding on a therapeutic formulation suitable for specific immunomodulation away from of the individual's current unwanted immune response.

16. A formulation that is immunoneutral to the immune system of an individual and capable of solubilizing hydrophobic antigens.

17. A formulation according to claim 3 where immunoneutral hydrophobic solvent is species specific Serum Albumin.

18. A formulation according to claim 4 including an antigen for stimulation of the immune system.

19. A formulation for antigen specific downregulation of inflammatory responses comprising at least two of the ingredients antigen and alum.

20. A formulation according to claim 5 or 6, where the antigen is of at least one from the group of ICA512 (IA2), ICA512B (IA2B), insulin, insulin B-chain, proinsulin, Hsp60, Hsp65, P277, ICA69, Glima38, GAD 65, GAD67, SOX13, Imogen 38, Sulfatide, MBP, MOG, Collagen II, 21-Ohase, TPO, allergens, transplant antigens, cancer antigens, or parts, peptides or altered peptide ligands thereof.

21. A kit for evaluation of lymphocyte reaction to antigen comprising a formulation according to claim 7.

22. A therapeutic comprising a formulation according to claim 7.

23. A therapeutic according to claim 9 where concentrations of antigen for in vitro stimulation typically is between 1-100 micrograms per ml.

24. A therapeutic according to claim 9 where concentrations of antigen for in vitro stimulation typically is between 5-40 micrograms per ml.

25. A therapeutic according to claim 9 where the subcutaneous administration of antigen for in vivo stimulation typically is between 0.1 micrograms per ml.

26. A therapeutic according to claim 9 where intravenous administration of antigen for in vivo stimulation typically is between 0.1-5 mgs/kg.
